(19) United States
(12) Patent Application Publication
(10) Pub. No.: US 2014/0215655 A1
(43) Pub. Date: Jul. 31, 2014

(54) GUARD CELL-SPECIFIC EXPRESSION OF TRANSGENES IN COTTON

(71) Applicants: Stéphane Pien, Bergisch Gladbach (DE); Bart Den Boer, Merelbeke (BE)

(72) Inventors: Stéphane Pien, Bergisch Gladbach (DE); Bart Den Boer, Merelbeke (BE)

(73) Assignee: BAYER CROPSCIENCE NV, Diegem (BE)

(21) Appl. No.: 14/238,669

(22) PCT Filed: Aug. 9, 2012
(86) PCT No.: PCT/EP2012/065608

§ 371 Date: Feb. 12, 2014

Related U.S. Application Data
(60) Provisional application No. 61/522,796, filed on Aug. 12, 2011.

(30) Foreign Application Priority Data
Aug. 12, 2011 (EP) 11075193.0

(51) Int. Cl. C12N 15/82

(52) U.S. Cl. CPC C12N 15/8273
USPC 800/287; 435/419; 435/6.11

(57) ABSTRACT

In one aspect, the application discloses a cotton plant cell comprising (a) a chimeric gene comprising a first nucleic acid sequence comprising at least 700 consecutive nucleotides of SEQ ID NO: 1 or a nucleic acid sequence having at least 80% sequence identity thereto any of which has stomata-preferential promoter activity; (b) a second nucleic acid sequence encoding an expression product of interest; and (c) a transcription termination and polyadenylation sequence. In addition, the present application discloses a cotton plant, a method of expressing a transgene in cotton under stress conditions, a method of producing a cotton plant, a method of detecting the expression of a transgene under stress conditions and a method for modulating the resistance of a cotton plant to stress as characterized in the claims.

In one aspect, the present application discloses a cotton plant cell comprising (a) a chimeric gene comprising a first nucleic acid sequence comprising at least 700 consecutive nucleotides of SEQ ID NO: 1 or a nucleic acid sequence having at least 80% sequence identity thereto any of which has stomata-preferential promoter activity; (b) a second nucleic acid sequence encoding an expression product of interest; and (c) a transcription termination and polyadenylation sequence. In addition, the present application discloses a cotton plant, a method of expressing a transgene in cotton under stress conditions, a method of producing a cotton plant, a method of detecting the expression of a transgene under stress conditions and a method for modulating the resistance of a cotton plant to stress as characterized in the claims.

In recent years the phenomenon of global warming, decreased fresh water supply and the effect of both on crop plant production has become a crucial issue. Solving this problem at the plant science level is almost exclusively a question of coping with plant stress. International agricultural and environmental research institutions now re-discover plant stress as a major component of the effect of global warming on local and global food production. Research to meet these challenges involves learning in widely diverging disciplines such as atmospheric sciences, soil science, plant physiology, biochemistry, genetics, plant breeding, molecular biology, and agricultural engineering.

Abiotic plant environmental stress constitutes a major limitation to crop production. The major plant environmental stresses of contemporary economical importance worldwide are water stress including drought and flooding, cold (chilling and freezing), heat, salinity, water logging, soil mineral deficiency, soil mineral toxicity and oxidative stress. These factors are not isolated but also interrelated and influencing each other.

Abscisic acid (ABA) is a phytohormone which functions in many plant developmental processes, including bud dormancy. Furthermore, ABA mediates stress responses in plants in reaction to water stress, high-salt stress, cold stress (Mansfield 1987, Yamaguchi-Shinozaki 1993, Yamaguchi-Shinozaki 1994) and plant pathogens (Seo and Koshiba, 2002). ABA is a sesquiterpenoid (15-carbon) which is partially produced via the mevalonic pathway in chloroplasts and other plastids. It is synthesized partially in the chloroplasts and accordingly, biosynthesis primarily occurs in the leaves. The production of ABA is increased by stresses such as water loss and freezing temperatures. It is believed that biosynthesis occurs indirectly through the production of carotenoids.

Physiological responses known to be associated with abscisic acid include stimulation of the closure of stomata, inhibition of shoot growth, induction of storage protein synthesis in seeds and inhibition of the effect of gibberellins on stimulating de novo synthesis of α-amylase.

Basic ABA levels may differ considerably from plant to plant. For example, the basal concentration of ABA in non-stressed Arabidopsis leaves is 2 to 3 ng/g fresh weight (Lopez-Carbonell and Jauregui, 2005). Under water-stress conditions, the ABA concentration reaches 10 to 21 ng/g fresh weight. On the other hand, in non-stressed cotton, the concentration of ABA in leaves varies between 145 to 2490 ng/g fresh weight (Ackerson, 1982).

Guard cells are located in the leaf epidermis and pair wise surround stomatal pores, which allow CO₂ influx for photosynthetic carbon fixation and water loss via transpiration to the atmosphere. Signal transduction mechanisms in guard cells integrate a multitude of different stimuli to modulate stomatal aperture. Stomata open in response to light. In response to drought stress, plants synthesize ABA which triggers closing of stomatal pores. The transport of ions and water through channel proteins across the plasma and vacuolar membranes changes turgor and guard cell volume, thereby regulating stomatal aperture (Pandey et al., 2007; Schroeder et al., 2001; Kim et al., 2010).

A major challenge in agriculture practice and research today is how to cope with plant environmental stress in an economical and environmentally sustainable approach. In view of the already existing regions exposed to abiotic stress and the ongoing climate change, the provision of transgenic plants improving or conferring tolerance to at least one kind of abiotic stress is still a major goal in order to achieve a satisfying nutritional situation also in regions exposed to such abiotic stress in the world.

Accordingly, in one aspect, the present application discloses a cotton plant cell comprising a chimeric gene comprising (a) a first nucleic acid sequence comprising at least 700 consecutive nucleotides of SEQ ID NO: 1 or a nucleic acid sequence having at least 80% sequence identity thereto any of which has stomata-preferential or stomata-specific promoter activity; (b) a second nucleic acid sequence encoding an expression product of interest; and (c) a transcription termination and polyadenylation sequence.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

“Cotton” or “cotton plant” as used herein includes Gossypium species such as Gossypium hirsutum, Gossypium barbadense, Gossypium arboreum and Gossypium herbaceum or progeny from crosses of such species with other species or crosses between such species.

A cotton plant cell may be any cell comprising essentially the genetic information necessary to define a cotton plant, which may, apart from the chimeric gene disclosed herein, be supplemented by one or more further transgenes. Cells may be derived from the various organs and/or tissues forming a cotton plant, including but not limited to fruits, seeds, embryos, reproductive tissue, meristematic regions, callus tissue, leaves, roots, shoots, flowers, vascular tissue, gametophytes, sporophytes, pollen, and microspores.

Unless indicated otherwise, the embodiments described below for the chimeric gene disclosed herein are also applicable to respective embodiments of other aspects disclosed herein.

As used herein, the term “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein or attached to another nucleic acid or protein stretch. A chimeric gene comprising a DNA region which is functionally or structurally defined may accordingly comprise additional DNA regions etc. However, in context with the present disclosure, the term “comprising” also includes “consisting of”.

A chimeric gene is an artificial gene constructed by operably linking fragments of unrelated genes or other nucleic acid sequences. In other words “chimeric gene” denotes a gene which is not normally found in a plant species or refers to any gene in which the promoter or one or more other regulatory regions of the gene are not associated in nature with a part or all of the transcribed nucleic acid, i.e. are heterologous with respect to the transcribed nucleic acid. More particularly, a chimeric gene is an artificial, i.e. non-naturally occurring, gene produced by an operable linkage of the first nucleic acid sequence comprising at least 700 consecutive nucleotides of SEQ ID NO: 1 or a nucleic acid sequence having at least 80% sequence identity thereto any of which has stomata-preferential or stomata-specific promoter activity with the second nucleic acid sequence encoding an expression product of interest which is not naturally operably linked to said nucleic acid sequence.

The present invention also relates to the chimeric gene as described above for use in cotton.

The term “heterologous” refers to the relationship between two or more nucleic acid or protein sequences that are derived from different sources. For example, a promoter is heterologous with respect to an operably linked nucleic acid sequence, such as a coding sequence, if such a combination is not normally found in nature. In addition, a particular sequence may be “heterologous” with respect to a cell or organism into which it is inserted (i.e. does not naturally occur in that particular cell or organism). For example, the chimeric gene disclosed herein is a heterologous nucleic acid.

Nucleic acids can be DNA or RNA, single- or double-stranded. Nucleic acids can be synthesized chemically or produced by biological expression in vitro or even in vivo.

Nucleic acids can be chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK).

In connection with the chimeric gene of the present disclosure, DNA includes cDNA and genomic DNA.

Said first nucleic acid sequence has stomata-preferential promoter activity. In other words, expression of said chimeric gene is preferentially induced in the stomata of a plant comprising said chimeric gene.

A promoter is stomata-preferential if transcription of a nucleic acid sequence controlled by said promoter is at least 5 times higher, at least 10 times higher, at least 20 times higher or at least 50 times higher in a guard cell than in cells of any other plant tissue. For the present invention, the promoter may also be stomata-specific. “Stomata-specific” expression (or “transcription” which is equivalent) in the context of this invention means the transcription of a nucleic acid sequence by a transcription regulating element such as a promoter in a way that transcription of said nucleic acid sequence in guard cells contributes to more than 50%, preferably more than 60%, more preferably more than 70%, even more preferably more than 80% of the entire quantity of the RNA transcribed from said nucleic acid sequence in the entire plant during any of its developmental stages.

Accordingly, the length of the first nucleic acid sequence and its position within SEQ ID NO: 1 is to be chosen such that it is sufficiently long and positioned such that expression of the chimeric gene comprising it is induced preferentially or specifically in guard cells (stomata).

Confirmation of, in this case stomata-preferential or stomata-specific, promoter activity for a promoter sequence or a functional promoter fragment may be determined by those skilled in the art, for example using a promoter-reporter construct comprising the promoter sequence described herein operably linked to an easily scorable marker as herein further explained. Such easily scorable marker, e.g. in the form of a reporter gene, can be e.g. a beta-glucuronidase (GUS) gene, chloramphenicol acetyl transferase (CAT) gene, beta-galactosidase (beta-GAL) gene, and genes encoding proteins with fluorescent or phosphorescent properties, such as green fluorescent protein (GFP) from Aequora Victoria or luciferase. Subsequently, such a chimeric gene is introduced into a plant and the expression pattern of the marker in stomata as compared to the expression pattern of the marker in other parts of the plant is analyzed. To e.g. define a minimal promoter, a nucleic acid sequence representing the promoter is operably linked to the coding sequence of a marker (reporter) gene by recombinant DNA techniques well known to the art. The reporter gene is operably linked downstream of the promoter, so that transcripts initiating at the promoter proceed through the reporter gene. The expression cassette containing the reporter gene under the control of the promoter can be introduced into an appropriate cell type by transformation techniques well known in the art and described elsewhere in this application. To assay for the reporter protein, cell lysates are prepared and appropriate assays, which are well known in the art, for the reporter protein are performed. For example, if CAT were the reporter gene of choice, the lysates from cells transfected with constructs containing CAT under the control of a promoter under study are mixed with isotopically labeled chloramphenicol and acetyl-coenzyme A (acetyl-CoA). The CAT enzyme transfers the acetyl group from acetyl-CoA to the 2- or 3-position of chloramphenicol. The reaction is monitored by thin-layer chromatography, which separates acetylated chloramphenicol from unreacted material. The reaction products are then visualized by autoradiography. The level of enzyme activity corresponds to the amount of enzyme that was made, which in turn reveals the level of expression and the stomata-preferential or stomata-specific functionality of the promoter or fragment or variant thereof in different cells or tissues. This level of expression can also be compared to other promoters to determine the relative strength of the promoter under study. Once activity and functionality is confirmed, additional mutational and/or deletion analyses may be employed to determine e.g. a minimal region and/or sequences required to initiate transcription. Thus, sequences can be deleted at the 5 end of the promoter region and/or at the 3′ end of the promoter region, or within the promoter sequence and/or nucleotide substitutions may be introduced. These constructs are then again introduced into cells and their activity and/or functionality are determined as described above.

Instead of measuring the activity of a reporter enzyme, the transcriptional promoter activity (and functionality) in different cell types or tissues can also be determined by measuring the level of RNA that is produced. This level of RNA, such as mRNA, can be measured either at a single time point or at multiple time points and as such the fold increase can be average fold increase or an extrapolated value derived from experimentally measured values. As it is a comparison of levels, any method that measures mRNA levels can be used. In an example, the tissue or organs compared are stomata with a leaf or leaf tissue, both preferably without stomata. In another example, multiple tissues or organs are compared. One example for multiple comparisons is stomata compared with 2, 3, 4, or tissues or organs selected from the group consisting of floral tissue, floral apex, pollen, leaf (preferably without stomata), embryo, shoot, leaf primordia, shoot apex, root, root tip, vascular tissue and cotyledon. As used herein, examples of plant organs are seed, leaf, root, etc. and examples of tissues are leaf primordia, shoot apex, vascular tissue, etc. The activity or strength of a promoter may be measured in terms of the amount of mRNA or protein accumulation it specifically produces, relative to the total amount of mRNA or protein.

Said first nucleic acid sequence having stomata-preferential or stomata-specific promoter activity in some examples may accordingly comprise at least 700, at least 800, at least 900, at least 1000, at least 1100, at least 1200, at least 1300, at least 1400, at least 1500 or at least 1600 consecutive nucleotides of SEQ ID NO: 1. In another example, said first nucleic acid sequence comprises the nucleotide sequence of SEQ ID NO: 1. In yet another example, said first nucleic acid sequence consists of SEQ ID NO: 1.

In one example, the first nucleic acid as described above comprises the 3′ end of SEQ ID NO: 1. Said 3′ end comprises at least the first 100 bases, at least the first 200 bases, at least the first 300 bases, at least the first 400 bases, at least the first 500 bases, at least the first 600 bases, at least the first 700 bases, at least the first 800 bases, at least the first 900 bases, at least the first 1000 bases, at least the first 1100 or at least the first 1200 bases of SEQ ID NO: 1.

In one aspect, nucleic acid sequences for promoters having stomata-preferential or stomata-specific activity comprising a nucleotide sequence having at least 70%, at least 80%, at least 90%, at least 95% or at least 98% sequence identity to SEQ ID NO: 1 are provided. Such nucleic acid sequences also include artificially derived nucleic acid sequences, such as those generated, for example, by using site-directed mutagenesis of SEQ ID NO: 1. Generally, nucleotide sequence variants disclosed herein may have at least 70%, such as 72%, 74%, 76%, 78%, at least 80%, e.g., 81% to 84%, at least 85%, e.g., 86%, 87%, 88%, 89%, 90%, 91%, 92%, 93%, 94%, 95%, 96%, 97%, to 98%, and 99% sequence identity to the nucleic acid sequence of SEQ ID NO: 1.

As used herein, the term “percent sequence identity” refers to the percentage of identical nucleotides between two segments of a window of optimally aligned DNA. Optimal alignment of sequences for aligning a comparison window are well-known to those skilled in the art and may be conducted by tools such as the local homology algorithm of Smith and Waterman (Waterman, M. S., Chapman & Hall. London, 1995), the homology alignment algorithm of Needleman and Wunsch (1970), the search for similarity method of Pearson and Lipman (1988), and preferably by computerized implementations of these algorithms such as GAP, BESTFIT, FASTA, and TFASTA available as part of the GCG (Registered Trade Mark), Wisconsin Package (Registered Trade Mark from Accelrys Inc., San Diego, Calif.). An “identity fraction” for aligned segments of a test sequence and a reference sequence is the number of identical components that are shared by the two aligned sequences divided by the total number of components in the reference sequence segment, i.e., the entire reference sequence or a smaller defined part of the reference sequence. Percent sequence identity is represented as the identity fraction times 100. The comparison of one or more DNA sequences may be to a full-length DNA sequence or a portion thereof, or to a longer DNA sequence. Sequence identity is calculated based on the shorter nucleotide sequence.

Only first nucleic acid sequences with the above-indicated degree of sequence identity which have stomata-preferential or stomata-specific promoter activity are encompassed by the present invention. Nucleic acid sequences representing stomata-preferential or stomata-specific promoters disclosed herein may also include, but are not limited to, deletions of sequence, single or multiple point mutations, alterations at a particular restriction enzyme recognition site, addition of functional elements, or other means of molecular modification which may enhance, or otherwise alter promoter expression as long as stomata-preferential or stomata-specific activity is essentially retained. Techniques for obtaining such derivatives are well-known in the art (see, for example, J. F. Sambrook, D. W. Russell, and N. Irwin, 2000). For example, one of ordinary skill in the art may delimit the functional elements within the promoters disclosed herein and delete any non-essential elements. The functional elements may be modified or combined to increase the utility or expression of the sequences of the invention for any particular application. Those of skill in the art are familiar with the standard resource materials that describe specific conditions and procedures for the construction, manipulation, and isolation of macromolecules (e.g. DNA molecules, plasmids, etc.), as well as the generation of recombinant organisms and the screening and isolation of DNA molecules.

The promoter sequence of at least 700 consecutive nucleotides of SEQ ID NO: 1 and its variants as described herein may for example be altered to contain e.g. “enhancer DNA” to assist in elevating gene expression. As is well-known in the art, certain DNA elements can be used to enhance the transcription of DNA. These enhancers are often found 5′ to the start of transcription in a promoter that functions in eukaryotic cells, but can often be inserted upstream (5′) or downstream (3′) to the coding sequence. In some instances, these enhancer DNA elements are introns. Among the introns that are useful as enhancer DNA are the 5′ introns from the rice actin 1 gene (see U.S. Pat. No. 5,641,876), the rice actin 2 gene, the Arabidopsis histone 4 intron, the maize alcohol dehydrogenase gene, the maize heat shock protein 70 gene (see U.S. Pat. No. 5,593,874), the maize shrunken 1 gene, the light sensitive 1 gene of Solanum tuberosum, and the heat shock protein 70 gene of Petunia hybrida (see U.S. Pat. No. 5,659,122). Thus, as contemplated herein, a promoter or promoter region includes variations of promoters derived by inserting or deleting regulatory regions, subjecting the promoter to random or site-directed mutagenesis etc. The activity or strength of a promoter may be measured in terms of the amounts of RNA it produces, or the amount of protein accumulation in a cell or tissue, relative to a promoter whose transcriptional activity has been previously assessed, as described above.

An expression product denotes an intermediate or end product arising from the transcription and optionally translation of the nucleic acid, DNA or RNA, coding for such product, e.g. the second nucleic acid described herein. During the transcription process, a DNA sequence under control of regulatory regions, particularly the promoter, is transcribed into an RNA molecule. An RNA molecule may either itself form an expression product or be an intermediate product when it is capable of being translated into a peptide or protein. A gene is said to encode an RNA molecule as expression product when the RNA as the end product of the expression of the gene is, e.g., capable of interacting with another nucleic acid or protein. Examples of RNA expression products include inhibitory RNA such as e.g. sense RNA (co-suppression), antisense RNA, ribozymes, miRNA or siRNA, mRNA, rRNA and tRNA. A gene is said to encode a protein as expression product when the end product of the expression of the gene is a protein or peptide.

Within the scope of the present disclosure, use may also be made, in combination with the first and second nucleic acid sequence described above, of other regulatory sequences, which are located between said first nucleic acid sequence comprising a promoter and said second nucleic acid sequence comprising the coding sequence of the expression product. Non-limiting examples of such regulatory sequences include transcription activators (“enhancers”), for instance the translation activator of the tobacco mosaic virus (TMV) described in Application WO 87/07644, or of the tobacco etch virus (TEV) described by Carrington & Freed 1990, J. Virol. 64: 1590-1597, or introns as described elsewhere in this application. Other suitable regulatory sequences include 5′ UTRs. As used herein, a 5′UTR, also referred to as leader sequence, is a particular region of a messenger RNA (mRNA) located between the transcription start site and the start codon of the coding region. It is involved in mRNA stability and translation efficiency. For example, the 5′ untranslated leader of a petunia chlorophyll a/b binding protein gene downstream of the 35S transcription start site can be utilized to augment steady-state levels of reporter gene expression (Harpster et al., 1988, Mol Gen Genet. 212(1):182-90). WO95/006742 describes the use of 5 non-translated leader sequences derived from genes coding for heat shock proteins to increase transgene expression.

The chimeric gene may also comprise a transcription termination or polyadenylation sequence operable in a plant cell, particularly a cotton plant cell. As a transcription termination or polyadenylation sequence, use may be made of any corresponding sequence of bacterial origin, such as for example the nos terminator of Agrobacterium tumefaciens, of viral origin, such as for example the CaMV 35S terminator, or of plant origin, such as for example a histone terminator as described in published Patent Application EP 0 633 317 A1.

The nucleotide sequence of SEQ ID NO: 1 represents a promoter found in Arabidopsis (Yang et al., 2008) in 3′-5′ direction. The promoter comprises at least two types of cis-acting elements one of which, ABRE-like, is involved in the ABA-associated stress response and the other one represents the motif (T/A)AAAG that has been shown to contribute to guard-cell specific gene expression.

It has been shown in the course of the present invention that a nucleic acid sequence of SEQ ID NO: 1 corresponding to the GC promoter (pGC) from Arabidopsis thaliana (Yang et al., Plant Methods 2008, 4:6) is sufficient to activate transcription of operably linked genes preferentially or specifically in the stomata of cotton leaves.

The abundant concentration of ABA in some plants might lead to a constitutive induction of an ABA-responsive promoter thus preventing specific expression of said promoter in certain tissues or organs.

The basal concentration of ABA in Arabidopsis leaves is 2-3 ng/g fresh weight (Löpez-Carbonell and Jäuregui, 2005). Under drought-stress conditions, the ABA concentration reaches 10-21 ng g-1 fresh weight (f.w.). However, in non-stressed cotton plants, the ABA concentration in leaves already varies between 145 to 2490 ng g-1 f.w. (Ackerson, 1982).

This range of concentrations in cotton would be expected to permanently activate promoters responsive to ABA when introduced in cotton.

The present inventors generated transgenic cotton plants using the GUS reporter under the control of the GCI promoter comprising an ABA-responsive element (ABRE). In the course of the present invention it was surprisingly found that this promoter region triggers GUS expression specifically in the stomata of the leaves of cotton plants. Surprisingly, despite the high endogenous level of ABA in cotton leaves, the activity of the GCI promoter is induced selectively in stomata.

The utility of the chimeric genes described above as well as of the various other aspects disclosed herein will be described below. For example, the disclosure of the present application can be used to modulate stomatal aperture in cotton plants, for example in order to facilitate growing cotton plants in regions where cotton plants are exposed to one or more kinds of abiotic stress at least once in their lifetime.

In one example of the cotton plant cell as described herein said expression product of interest is (i) a protein or peptide, optionally involved in mediating stomatal aperture or (ii) an RNA molecule capable of modulating the expression of a gene comprised in said cotton plant, wherein optionally said gene comprised in said cotton plant is involved in mediating stomatal aperture. A gene comprised in a cotton plant may be endogenous to the cotton plant or have been introduced into said cotton plant. The latter in particular applies to target expression products which are not endogenous to cotton plants or to homologues of expression products endogenous in cotton plants but derived from other organisms.

The term “protein” as used herein describes a group of molecules consisting of more than 30 amino acids, whereas the term “peptide” describes molecules consisting of up to 30 amino acids. Proteins and peptides may further form dimers, trimers and higher oligomers, i.e. consisting of more than one (poly)peptide molecule. Protein or peptide molecules forming such dimers, trimers etc. may be identical or non-identical. The corresponding higher order structures are, consequently, termed homo- or heterodimers, homo- or heterotrimers etc. The terms “protein” and “peptide” also refer to naturally modified proteins or peptides wherein the modification is effected e.g. by glycosylation, acetylation, phosphorylation and the like. Such modifications are well known in the art.

Said expression product of interest may also be an RNA molecule capable of modulating the expression of a gene comprised in said cotton plant, wherein said gene is optionally involved in mediating stomatal aperture.

Example proteins and nucleic acids, such as genes, optionally involved in mediating stomatal aperture, suitable as expression products include:

Examples of genes involved in mediating stomatal aperture include those encoding carbonic anhydrase, OST1, HT1 or proteins involved in mediating ABA responsiveness.

For the case of RNA molecules, it will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

The term “capable of modulating the expression of a gene” relates, inter alia, to the action of an RNA molecule, such as an inhibitory RNA molecule as described herein, to influence the expression level of target genes in different ways. This can be effected by inhibiting the expression of a target gene by directly interacting with components driving said expression such as the gene itself or the transcribed mRNA which results in a decrease of expression, or another gene involved in inhibiting the expression of a gene, wherein said gene is optionally involved in the mediation of stomatal aperture.

Inhibitory RNA molecules decrease the levels of mRNAs of their target expression products such as target proteins available for translation into said target protein. In this way, expression of proteins, for example those involved in stomatal opening or closing (aperture), can be inhibited. This can be achieved through well established techniques including co-suppression (sense RNA suppression), antisense RNA, double-stranded RNA (dsRNA), or microRNA (miRNA).

An RNA molecule as expression product as disclosed herein comprises a part of a nucleotide sequence encoding a target expression product such as target protein or RNA or a homologous sequence to down-regulate the expression of said target expression product. Another example for an RNA molecule as expression product for use in down-regulating expression are antisense RNA molecules comprising a nucleotide sequence complementary to at least a part of a nucleotide sequence encoding an expression product such as a protein or RNA of interest or a homologous sequence. Here, down-regulation may be effected e.g. by introducing this antisense RNA or a chimeric DNA encoding such RNA molecule. In yet another example, expression of an expression product of interest such as a protein or RNA of interest is down-regulated by introducing a double-stranded RNA molecule comprising a sense and an antisense RNA region corresponding to and respectively complementary to at least part of a gene sequence encoding said expression product of interest, which sense and antisense RNA region are capable of forming a double stranded RNA region with each other. Such double-stranded RNA molecule may be encoded both by sense and antisense molecules as described above and by a single-stranded molecule being processed to form siRNA (as described e.g. in EP1583832) or miRNA.

In one example, expression of a target protein may be down-regulated by introducing a chimeric DNA construct which yields a sense RNA molecule capable of down-regulating expression by co-suppression. The transcribed DNA region will yield upon transcription a so-called sense RNA molecule capable of reducing the expression of a gene encoding a target expression product such as a target protein or RNA in the target plant or plant cell in a transcriptional or post-transcriptional manner. The transcribed DNA region (and resulting RNA molecule) comprises at least 20 consecutive nucleotides having at least 95% sequence identity to the corresponding portion of the nucleotide sequence encoding the target expression product such as a target protein present in the plant cell or plant.

Alternatively, an expression product for down-regulating expression of a target expression product such as a target protein or RNA is an antisense RNA molecule. Down-regulating or reducing the expression of an expression product of interest in the target cotton plant or plant cell is effected in a transcriptional or post-transcriptional manner. The transcribed DNA region (and resulting RNA molecule) comprises at least 20 consecutive nucleotides having at least 95% sequence identity to the complement of the corresponding portion of the nucleic acid sequence encoding said target expression product present in the plant cell or plant.

However, the minimum nucleotide sequence of the antisense or sense RNA region of about 20 nt of the nucleic acid sequence encoding a target expression product may be comprised within a larger RNA molecule, varying in size from 20 nt to a length equal to the size of the target gene. The mentioned antisense or sense nucleotide regions may thus be about from about 21 nt to about 5000 nt long, such as 21 nt, 40 nt, 50 nt, 100 nt, 200 nt, 300 nt, 500 nt, 1000 nt, 2000 nt or even about 5000 nt or larger in length. Moreover, it is not required for the purpose of the invention that the nucleotide sequence of the used inhibitory RNA molecule or the encoding region of the transgene, is completely identical or complementary to the target gene, which may be endogenous to the plant or have been introduced, encoding the target expression product the expression of which is targeted to be reduced in the plant cell. The longer the sequence, the less stringent the requirement for the overall sequence identity is. Thus, the sense or antisense regions may have an overall sequence identity of about 40% or 50% or 60% or 70% or 80% or 90% or 100% to the nucleotide sequence of the target gene or the complement thereof. However, as mentioned, antisense or sense regions should comprise a nucleotide sequence of 20 consecutive nucleotides having about 95 to about 100% sequence identity to the nucleotide sequence encoding the target gene. The stretch of about 95 to about 100% sequence identity may be about 50, 75 or 100 nt.

The efficiency of the above mentioned chimeric genes for antisense RNA or sense RNA-mediated gene expression level down-regulation may be further enhanced by inclusion of DNA elements which result in the expression of aberrant, non-polyadenylated inhibitory RNA molecules. One such DNA element suitable for that purpose is a DNA region encoding a self-splicing ribozyme, as described in WO 00/01133. The efficiency may also be enhanced by providing the generated RNA molecules with nuclear localization or retention signals as described in WO 03/076619.

In addition, an expression product as described herein may be a nucleic acid sequence which yields a double-stranded RNA molecule capable of down-regulating expression of a gene encoding a target expression product. Upon transcription of the DNA region the RNA is able to form dsRNA molecule through conventional base paring between a sense and antisense region, whereby the sense and antisense region are nucleotide sequences as hereinbefore described. Expression products being dsRNA according to the invention may further comprise an intron, such as a heterologous intron, located e.g. in the spacer sequence between the sense and antisense RNA regions in accordance with the disclosure of WO 99/53050. To achieve the construction of such a transgene, use can be made of the vectors described in WO 02/059294 A1.

In an example, said RNA molecule comprises a first and second RNA region wherein 1. said first RNA region comprises a nucleotide sequence of at least 19 consecutive nucleotides having at least about 94% sequence identity to the nucleotide sequence of said gene comprised in said cotton plant; 2. said second RNA region comprises a nucleotide sequence complementary to said 19 consecutive nucleotides of said first RNA region; 3. said first and second RNA region are capable of base-pairing to form a double stranded RNA molecule between at least said 19 consecutive nucleotides of said first and second region.

Another example expression of interest product is a microRNA molecule (miRNA, which may be processed from a pre-microRNA molecule) capable of guiding the cleavage of mRNA transcribed from the DNA encoding the target expression product, such as a protein or an RNA, which is to be translated into said target expression product. miRNA molecules or pre-miRNA molecules may be conveniently introduced into plant cells through expression from a chimeric gene as described herein comprising a (second) nucleic acid sequence encoding as expression product of interest such miRNA, pre-miRNA or primary miRNA transcript. miRNAs are small endogenous RNAs that regulate gene expression in plants, but also in other eukaryotes. As used herein, a “miRNA” is an RNA molecule of about 19 to 22 nucleotides in length which can be loaded into a RISC complex and direct the cleavage of a target RNA molecule, wherein the target RNA molecule comprises a nucleotide sequence essentially complementary to the nucleotide sequence of the miRNA molecule. In one example, one or more of the following mismatches may occur in the essentially complementary sequence of the miRNA molecule:
A mismatch between the nucleotide at the 5′ end of said miRNA and the corresponding nucleotide sequence in the target RNA molecule;
A mismatch between any one of the nucleotides in position 1 to position 9 of said miRNA and the corresponding nucleotide sequence in the target RNA molecule;
Three mismatches between any one of the nucleotides in position 12 to position 21 of said miRNA and the corresponding nucleotide sequence in the target RNA molecule provided that there are no more than two consecutive mismatches;
No mismatch is allowed at positions 10 and 11 of the miRNA (all miRNA positions are indicated starting from the 5′ end of the miRNA molecule).

As used herein, a “pre-miRNA” molecule is an RNA molecule of about 100 to about 200 nucleotides, preferably about 100 to about 130 nucleotides which can adopt a secondary structure comprising a dsRNA stem and a single stranded RNA loop and further comprising the nucleotide sequence of the miRNA and its complement sequence of the miRNA* in the double-stranded RNA stem. Preferably, the miRNA and its complement are located about 10 to about 20 nucleotides from the free ends of the miRNA dsRNA stem. The length and sequence of the single stranded loop region are not critical and may vary considerably, e.g. between 30 and 50 nt in length. Preferably, the difference in free energy between unpaired and paired RNA structure is between −20 and −60 kcal/mole, particularly around −40 kcal/mole. The complementarity between the miRNA and the miRNA* does not need to be perfect and about 1 to 3 bulges of unpaired nucleotides can be tolerated. The secondary structure adopted by an RNA molecule can be predicted by computer algorithms conventional in the art such as mFold, UNAFoId and RNAFoId. The particular strand of the dsRNA stem from the pre-miRNA which is released by DCL activity and loaded onto the RISC complex is determined by the degree of complementarity at the 5′ end, whereby the strand which at its 5′ end is the least involved in hydrogen bonding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional because the “wrong” strand is loaded on the RISC complex, it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds. miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds.

Example expression products can also be ribozymes catalyzing either their own cleavage or the cleavage of other RNAs.

In one example of the cotton plant cell disclosed herein modulating is increasing, mediating is inhibiting and said second nucleic acid sequence encodes an RNA, which when transcribed yields an RNA molecule capable of increasing the expression of a first gene comprised in said cotton plant, e.g. by targeting genes involved in down-regulating the expression of said first gene.

In another example of the cotton plant cell disclosed herein modulating is decreasing, mediating is activating and said second nucleic acid sequence encodes an RNA, which when transcribed 1. yields an RNA molecule capable of decreasing the expression of a first gene comprised in said cotton plant, e.g. by targeting genes involved in up-regulating the expression of said first gene, or 2. yields an RNA molecule capable of decreasing the expression of said gene comprised in said cotton plant, for example by targeting this gene directly.

Example RNA-based expression products include inhibitory RNAs such as miRNAs, siRNAs, antisense RNAs or ribozymes targeting enzymes of the PARP (poly(ADP-ribose) polymerase) family, examples of which are also disclosed in international patent application PCT/EP2010/003438.

Example genes to be targeted may encode proteins involved in the signal transduction pathway mediating stomatal aperture (see e.g. Hubbard et al., 2010).

In one example of the cotton plant cell described herein said first nucleic acid sequence of said chimeric gene comprises the nucleotide sequence of SEQ ID NO: 1 or a nucleic acid sequence having at least 70%, at least 80%, at least 90%, at least 95% or at least 98% sequence identity thereto and having stomata-preferential or stomata-specific promoter activity.

In another example of the cotton plant cell described herein said first nucleic acid sequence of said chimeric gene consists of SEQ ID NO: 1 or a nucleic acid sequence having at least 70%, at least 80%, at least 90%, at least 95% or at least 98% sequence identity thereto and having stomata-preferential or stomata-specific promoter activity.

It has been shown in the examples of this application that a transgene or chimeric gene can be efficiently expressed under the control of the GC1 promoter in guard cells (stomata) of a cotton plant. This enables for alleviating the effect of certain abiotic stress conditions such as drought for the plant by providing sequences encoding expression products which enhance water use efficiency. Such expression products will generally be those which inhibit stomatal opening or those inhibiting mechanisms involved in activating stomatal aperture.

Drought is one of the most serious world-wide problems for agriculture. Four-tenths of the world's agricultural land lies in arid or semi-arid regions. Transient droughts can cause death of livestock, famine and social dislocation. Other agricultural regions have consistently low rain-fall and rely on irrigation to maintain yields. In both circumstances, crop plants which can make the most efficient use of water and maintain acceptable yields will be at an advantage.

In another aspect, the present application discloses a cotton plant or seed thereof or plant part comprising (a) a chimeric gene comprising a. a first nucleic acid sequence comprising at least 700 consecutive nucleotides of SEQ ID NO: 1 or a nucleic acid sequence having at least 80% sequence identity thereto any of which has stomata-preferential or stomata-specific promoter activity; b. a second nucleic acid sequence encoding an expression product of interest; and c. a transcription termination and polyadenylation sequence; or (b) the cotton plant cell described herein. The chimeric gene described in (a) may be the chimeric gene as described herein above including all variations related thereto.

In some embodiments, the cotton plant cell described herein is a non-propagating plant cell or a plant cell that cannot be regenerated into a plant or a plant cell that cannot maintain its life by synthesizing carbohydrate and protein from the inorganics, such as water, carbon dioxide, and inorganic salt, through photosynthesis.

Seed is formed by an embryonic plant enclosed together with stored nutrients by a seed coat. It is the product of the ripened ovule of gymnosperm and angiosperm plants, to the latter of which cotton belongs, which occurs after fertilization and to a certain extent growth within the mother plant.

In another aspect, disclosed is a method of effecting stomata-preferential or stomata-specific expression of a product of interest in cotton: (a1) introducing a chimeric gene comprising a first nucleic acid sequence comprising at least 700 consecutive nucleotides of SEQ ID NO: 1 or a nucleic acid sequence having at least 80% sequence identity thereto any of which has stomata-preferential or stomata-specific promoter activity, a second nucleic acid sequence encoding an expression product of interest, and a transcription termination and polyadenylation sequence into a cotton plant and growing the plant; or (a2) growing the cotton plant described herein or growing a plant from the seed described herein. The chimeric gene described in (a1) may be the chimeric gene as described herein above including all variations related thereto.

“Introducing” in connection with the present application relates to the placing of genetic information in a plant cell or plant by artificial means. This can be effected by any method known in the art for introducing RNA or DNA into plant cells, tissues, protoplasts or whole plants. In addition, “introducing” also comprises introgressing genes as defined further below.

A number of methods are available to transfer DNA into plant cells. Agrobacterium-mediated transformation of cotton has been described e.g. in U.S. Pat. No. 5,004,863, in U.S. Pat. No. 6,483,013 and WO2000/71733.

Plants may also be transformed by particle bombardment: Particles of gold or tungsten are coated with DNA and then shot into young plant cells or plant embryos. This method also allows transformation of plant plastids. Cotton transformation by particle bombardment is reported e.g. in WO 92/15675.

Viral transformation (transduction) may be used for transient or stable expression of a gene, depending on the nature of the virus genome. The desired genetic material is packaged into a suitable plant virus and the modified virus is allowed to infect the plant. The progeny of the infected plants is virus free and also free of the inserted gene. Suitable methods for viral transformation are described or further detailed e.g. in WO 90/12107, WO 03/052108 or WO 2005/098004.

“Introgressing” means the integration of a gene in a plant's genome by natural means, i.e. by crossing a plant comprising the chimeric gene described herein with a plant not comprising said chimeric gene. The offspring can be selected for those comprising the chimeric gene.

Further transformation and introgression protocols can also be found in U.S. Pat. No. 7,172,881.

The chimeric gene may be introduced, e.g. by transformation, in cotton plants from which embryogenic callus can be derived, such as Coker 312, Coker310, Coker SAcala SJ-5, GSC25110, FIBERMAX 819, Siokra 1-3, T25, GSA75, Acala SJ2, Acala SJ4, Acala SJ5, Acala SJ-C1, Acala B1644, Acala B1654-26, Acala B1654-43, Acala B3991, Acala GC356, Acala GC510, Acala GAM1, Acala C1, Acala Royale, Acala Maxxa, Acala Prema, Acala B638, Acala B1810, Acala B2724, Acala B4894, Acala B5002, non Acala “picker” Siokra, “stripper” variety FC2017, Coker 315, STONEVILLE 506, STONEVILLE 825, DP50, DP61, DP90, DP77, DES119, McN235, HBX87, HBX191, HBX107, FC 3027, CHEMBRED A1, CHEMBRED A2, CHEMBRED A3, CHEMBRED A4, CHEMBRED B1, CHEMBRED B2, CHEMBRED B3, CHEMBRED C1, CHEMBRED C2, CHEMBRED C3, CHEMBRED C4, PAYMASTER 145, HS26, HS46, SICALA, PIMA S6 ORO BLANCO PIMA, FIBERMAX FM5013, FIBERMAX FM5015, FIBERMAX FM5017, FIBERMAX FM989, FIBERMAX FM832, FIBERMAX FM966, FIBERMAX FM958, FIBERMAX FM989, FIBERMAX FM958, FIBERMAX FM832, FIBERMAX FM991, FIBERMAX FM819, FIBERMAX FM800, FIBERMAX FM960, FIBERMAX FM966, FIBERMAX FM981, FIBERMAX FM5035, FIBERMAX FM5044, FIBERMAX FM5045, FIBERMAX FM5013, FIBERMAX FM5015, FIBERMAX FM5017 or FIBERMAX FM5024 and plants with genotypes derived thereof.

In a further aspect, the present application discloses a method of producing a cotton plant or of increasing the yield of a cotton plant comprising: introducing a chimeric gene comprising a first nucleic acid sequence comprising at least 700 consecutive nucleotides of SEQ ID NO: 1 or a nucleic acid sequence having at least 80% sequence identity thereto any of which has stomata-preferential or stomata-specific promoter activity, a second nucleic acid sequence encoding an expression product of interest, and a transcription termination and polyadenylation sequence; or growing the plant described herein or growing a plant from the seed disclosed herein. The chimeric gene introduced may be the chimeric gene as described herein above including all variations related thereto. The expression product of interest encoded by said second nucleic acid sequence comprised in said chimeric gene may be involved in the mediation of stomatal aperture as described elsewhere in this application.

Also disclosed herein is a method of growing cotton comprising (a1) providing the transgenic plant described herein or produced by the method of producing a cotton plant described herein; or (a2) introducing a chimeric gene described herein in a plant; (b) growing the plant of (al) or (a2); and (c) harvesting cotton produced by said plant.

Also disclosed herein is a method of detecting the expression of a transgene, comprising (a) providing the cotton plant cell or the plant disclosed herein, wherein said expression product of interest is the transgene and; (b) detecting the expression of the transgene.

The term “expression of a transgene” relates to the transcription and optionally the translation of the transcribable and optionally translatable part of the chimeric gene disclosed herein using appropriate expression control elements that function in cotton cells. As described above, the first nucleic acid sequence disclosed herein has stomata-preferential or stomata-specific promoter activity and is thus suitable to express an expression product of choice (corresponding to the second nucleic acid sequence) in the stomata of cotton.

“Detecting the expression of the transgene” can be effected in multiple ways. In case of the transgene being a reporter gene, expression of said reporter gene, depending on the feature rendering it a reporter gene, is easily detectable. For example if the reporter gene is an enzyme capable of converting a substrate into a visually detectable product, said product may be detected by the appropriate means which depend on the color of said product or of the wavelength of the light emitted by said product. In case the transgene is not a conventional reporter gene but has enzymatic activity, assays can be designed by the skilled person knowing said enzymatic activity to track and quantify it with suitable methods. Furthermore, expression of a transgene with known nucleic acid sequence can be measured by PCR methods including the one disclosed in Zanoni et al. (Nature 2009, 460, p:264-269, see also Nature Protocols: mRNA expression analysis by Real-Time PCR; ISSN: 1754-2189) and in Logan, Edwards and Saunders (Editors; Real-Time PCR: Current Technology and Applications, Caister Academic Press 2009, ISBN: 978-1-904455-39-4), by sequencing techniques including that disclosed in the Illumina datasheet “mRNA expression analysis” (2010) available at http://www.illumina.com/documents/products/datasheets/datasheet_mma_expression.pdf, and by hybridization techniques such as that disclosed in Chaudhary et al. (2008).

Also disclosed herein is a method for modulating the water use efficiency (WUE) of a cotton plant comprising introducing into a cotton plant a chimeric gene comprising a. a first nucleic acid sequence comprising at least 700 consecutive nucleotides of SEQ ID NO: 1 or a nucleic acid sequence having at least 80% sequence identity thereto any of which has stomata-preferential or stomata-specific promoter activity; b. a second nucleic acid sequence encoding an expression product of interest which is involved in the mediation of stomatal aperture; and c. a transcription termination and polyadenylation sequence; and growing said plant.

In one example of the method for modulating the water use efficiency of a cotton plant modulating is increasing. In this case, said second nucleic acid sequence may encode an RNA, which when transcribed 1. yields an RNA molecule capable of increasing the expression of a first gene comprised in said cotton plant which triggers stomatal closure, e.g. by targeting genes involved in down-regulating the expression of said first gene, or 2. yields an RNA molecule capable of decreasing the expression of a gene comprised in said cotton plant which triggers stomatal opening, for example by targeting this gene directly.

In another example of the method for modulating the water use efficiency of a cotton plant, modulating is decreasing. In this case, said second nucleic acid sequence may encode an RNA, which when transcribed 1. yields an RNA molecule capable of increasing the expression of a first gene comprised in said cotton plant which triggers stomatal opening, e.g. by targeting a gene involved in down-regulating the expression of said first gene, or 2. yields an RNA molecule capable of decreasing the expression of a gene comprised in said cotton plant which triggers stomatal closure, for example by targeting this gene directly.

Also disclosed herein is the use of (a) the cotton plant or seed disclosed herein; or (b) a chimeric gene comprising a. a first nucleic acid sequence comprising at least 700 consecutive nucleotides of SEQ ID NO: 1 or a nucleic acid sequence having at least 80% sequence identity thereto any of which has stomata-preferential or stomata-specific promoter activity; b. a second nucleic acid sequence encoding an expression product of interest; and c. a transcription termination and polyadenylation sequence; or (c) a nucleic acid sequence comprising at least 700 consecutive nucleotides of SEQ ID NO: 1 or a nucleic acid sequence having at least 80% sequence identity thereto any of which has stomata-preferential or stomata-specific promoter activity; for stomata-preferential or stomata-specific expression of a transgene in cotton, for modulating the water use efficiency of a cotton plant or for increasing cotton yield as described above. The chimeric gene utilized in this use may be the chimeric gene as described herein above in connection with the methods of the invention. Otherwise, all terms defining the present use have the meaning as described elsewhere in this application.

Also disclosed herein is the use of (a) a nucleic acid sequence comprising at least 700 consecutive nucleotides of SEQ ID NO: 1 or a nucleic acid sequence having at least 80% sequence identity thereto any of which has stomata-preferential or stomata-specific promoter activity; or (b) a chimeric gene comprising a. a first nucleic acid sequence comprising at least 700 consecutive nucleotides of SEQ ID NO: 1 or a nucleic acid sequence having at least 80% sequence identity thereto any of which has stomata-preferential or stomata-specific promoter activity; b. a second nucleic acid sequence encoding an expression product of interest; and c. a transcription termination and polyadenylation sequence; to detect a transgene in cotton fibers.

Further disclosed herein are cotton fibers and cotton seed oil obtainable or obtained from the plants disclosed herein. Cotton fibers disclosed herein can be distinguished from other fibers by applying the detection method disclosed in WO2010/015423 and checking for the presence of the nucleic acid of (a) or chimeric gene of (b) in the fibers.

Also disclosed herein are yarn and textiles made from the fibers disclosed herein as well as foodstuff and feed comprising or made of the cotton seed oil disclosed herein. A method to obtain cotton seed oil comprising harvesting cotton seeds from the cotton plant disclosed herein and extracting said oil from said seeds is also disclosed. Further, a method to produce cotton fibers comprising growing the cotton plant disclosed herein and harvesting cotton from said cotton plants is also disclosed.

Also disclosed herein is a method for alleviating the effect of drought on a cotton field comprising (a) obtaining cotton plants comprising (i) a chimeric gene comprising a. a first nucleic acid sequence comprising at least 700 consecutive nucleotides of SEQ ID NO: 1 or a nucleic acid sequence having at least 80% sequence identity thereto any of which has stomata-preferential or stomata-specific promoter activity; b. a second nucleic acid sequence encoding an expression product of interest; and c. a transcription termination and polyadenylation sequence; or progeny thereof; and (b) planting said cotton plants in said field.

The transformed cotton plant cells and cotton plants disclosed herein or obtained by the methods described herein may contain, in addition to the chimeric gene described above, at least one other chimeric gene comprising a nucleic acid encoding an expression product of interest. Examples of such expression product include RNA molecules or proteins, such as for example an enzyme for resistance to a herbicide. Herbicide-resistant cotton plants are for example glyphosate-tolerant plants, i.e. plants made tolerant to the herbicide glyphosate or salts thereof. Plants can be made tolerant to glyphosate through different means. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., 1983, Science 221, 370-371), the CP4 gene of the bacterium Agrobacterium sp. (Barry et al., 1992, Curr. Topics Plant Physiol. 7, 139-145), the genes encoding a Petunia EPSPS (Shah et al., 1986, Science 233, 478-481), a Tomato EPSPS (Gasser et al., 1988, J. Biol. Chem. 263, 4280-4289), or an Eleusine EPSPS (WO 01/66704). It can also be a mutated EPSPS as described in for example EP 0837944, WO 00/66746, WO 00/66747 or WO02/26995. Glyphosate-tolerant plants can also be obtained by expressing a gene that encodes a glyphosate oxido-reductase enzyme as described in U.S. Pat. Nos. 5,776,760 and 5,463,175. Glyphosate-tolerant plants can also be obtained by expressing a gene that encodes a glyphosate acetyl transferase enzyme as described in for example WO 02/36782, WO 03/092360, WO 05/012515 and WO 07/024782. Glyphosate-tolerant plants can also be obtained by selecting plants containing naturally-occurring mutations of the above-mentioned genes, as described in for example WO 01/024615 or WO 03/013226. Plants expressing EPSPS genes that confer glyphosate tolerance are described in e.g. U.S. patent application Ser. Nos 11/517,991, 10/739,610, 12/139,408, 12/352,532, 11/312,866, 11/315,678, 12/421,292, 11/400,598, 11/651,752, 11/681,285, 11/605,824, 12/468,205, 11/760,570, 11/762,526, 11/769,327, 11/769,255, 11/943801 or 12/362,774. Plants comprising other genes that confer glyphosate tolerance, such as decarboxylase genes, are described in e.g. U.S. patent application Ser. Nos. 11/588,811, 11/185,342, 12/364,724, 11/185,560 or 12/423,926.

Other herbicide resistant cotton plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition, e.g. described in U.S. patent application Ser. No. 11/760,602. One such efficient detoxifying enzyme is an enzyme encoding a phosphinothricin acetyltransferase (such as the bar or pat protein from Streptomyces species). Plants expressing an exogenous phosphinothricin acetyltransferase are for example described in U.S. Pat. Nos. 5,561,236; 5,648,477; 5,646,024; 5,273,894; 5,637,489; 5,276,268; 5,739,082; 5,908,810 and 7,112,665.

Further herbicide-tolerant cotton plants are also plants that are made tolerant to the herbicides inhibiting the enzyme hydroxyphenylpyruvatedioxygenase (HPPD). HPPD is an enzyme that catalyze the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated or chimeric HPPD enzyme as described in WO 96/38567, WO 99/24585, WO 99/24586, WO 2009/144079, WO 2002/046387, or U.S. Pat. No. 6,768,044. Tolerance to HPPD-inhibitors can also be obtained by transforming plants with genes encoding certain enzymes enabling the formation of homogentisate despite the inhibition of the native HPPD enzyme by the HPPD-inhibitor. Such plants and genes are described in WO 99/34008 and WO 02/36787. Tolerance of plants to HPPD inhibitors can also be improved by transforming plants with a gene encoding an enzyme having prephenate dehydrogenase (PDH) activity in addition to a gene encoding an HPPD-tolerant enzyme, as described in WO 2004/024928. Further, plants can be made more tolerant to HPPD-inhibitor herbicides by adding into their genome a gene encoding an enzyme capable of metabolizing or degrading HPPD inhibitors, such as the CYP450 enzymes shown in WO 2007/103567 and WO 2008/150473.

Still further herbicide resistant cotton plants are plants that are made tolerant to acetolactate synthase (ALS) inhibitors. Known ALS-inhibitors include, for example, sulfonylurea, imidazolinone, triazolopyrimidines, pryimidinyoxy(thio)benzoates, and/or sulfonylaminocarbonyltriazolinone herbicides. Different mutations in the ALS enzyme (also known as acetohydroxyacid synthase, AHAS) are known to confer tolerance to different herbicides and groups of herbicides, as described for example in Tranel and Wright (2002, Weed Science 50:700-712), but also, in U.S. Pat. Nos. 5,605,011, 5,378,824, 5,141,870, and 5,013,659. The production of sulfonylurea-tolerant plants and imidazolinone-tolerant plants is described in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270. Other imidazolinone-tolerant plants are also described in for example WO 2004/040012, WO 2004/106529, WO 2005/020673, WO 2005/093093, WO 2006/007373, WO 2006/015376, WO 2006/024351, and WO 2006/060634. Further sulfonylurea- and imidazolinone-tolerant plants are also described in for example WO 07/024782 and U.S. Patent Application No 61/288958.

Other cotton plants tolerant to imidazolinone and/or sulfonylurea can be obtained by induced mutagenesis, selection in cell cultures in the presence of the herbicide or mutation breeding as described for example for soybeans in U.S. Pat. No. 5,084,082, for rice in WO 97/41218, for sugar beet in U.S. Pat. No. 5,773,702 and WO 99/057965, for lettuce in U.S. Pat. No. 5,198,599, or for sunflower in WO 01/065922.

Further expression products of interest confer insect resistance to a cotton plant, i.e. resistance to attack by certain target insects. Such plants can be obtained by genetic transformation, or by selection of plants containing a mutation imparting such insect resistance.

Insect-resistant plants include any plant containing at least one transgene comprising a coding sequence encoding:

1) an insecticidal crystal protein from Bacillus thuringiensis or an insecticidal portion thereof, such as the insecticidal crystal proteins listed by Crickmore et al. (1998, Microbiology and Molecular Biology Reviews, 62: 807-813), updated by Crickmore et al. (2005) at the Bacillus thuringiensis toxin nomenclature, online at: http://www.lifesci.sussex.ac.uk/Home/Neil_Crickmore/Bt/), or insecticidal portions thereof, e.g., proteins of the Cry protein classes Cry1Ab, Cry1Ac, Cry1B, Cry1C, Cry1D, Cry1F, Cry2Ab, Cry3Aa, or Cry3Bb or insecticidal portions thereof (e.g. EP 1999141 and WO 2007/107302), or such proteins encoded by synthetic genes as e.g. described in and U.S. patent application Ser. No. 12/249,016; or
2) a crystal protein from Bacillus thuringiensis or a portion thereof which is insecticidal in the presence of a second other crystal protein from Bacillus thuringiensis or a portion thereof, such as the binary toxin made up of the Cry34 and Cry35 crystal proteins (Moellenbeck et al. 2001, Nat. Biotechnol. 19: 668-72; Schnepf et al. 2006, Applied Environm. Microbiol. 71, 1765-1774) or the binary toxin made up of the Cry1A or Cry1F proteins and the Cry2Aa or Cry2Ab or Cry2Ae proteins (U.S. patent application Ser. No. 12/214,022 and EP 08010791.5); or
3) a hybrid insecticidal protein comprising parts of different insecticidal crystal proteins from Bacillus thuringiensis, such as a hybrid of the proteins of 1) above or a hybrid of the proteins of 2) above, e.g., the Cry1A.105 protein produced by corn event MON89034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation, such as the Cry3Bb1 protein in corn events MON863 or MON88017, or the Cry3A protein in corn event MIR604; or
5) an insecticidal secreted protein from Bacillus thuringiensis or Bacillus cereus, or an insecticidal portion thereof, such as the vegetative insecticidal (VIP) proteins listed at: http://www.lifesci.sussex.ac.uk/home/Neil_Crickmore/Bt/vip.html, e.g., proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94/21795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 5) to 7) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein), such as the VIP3Aa protein in cotton event COT102; or
9) a secreted protein from Bacillus thuringiensis or Bacillus cereus which is insecticidal in the presence of a crystal protein from Bacillus thuringiensis, such as the binary toxin made up of VIP3 and Cry1A or Cry1F (U.S. Patent Appl. No. 61/126083 and 61/195019), or the binary toxin made up of the VIP3 protein and the Cry2Aa or Cry2Ab or Cry2Ae proteins (U.S. patent appliation Ser. No. 12/214,022 and EP 08010791.5);
10) a protein of 9) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein).

Also included are insect-resistant transgenic plants comprising a combination of genes encoding the proteins of any one of the above classes 1 to 10. In one embodiment, an insect-resistant plant contains more than one transgene encoding a protein of any one of the above classes 1 to 10, to expand the range of target insect species affected when using different proteins directed at different target insect species, or to delay insect resistance development to the plants by using different proteins insecticidal to the same target insect species but having a different mode of action, such as binding to different receptor binding sites in the insect.

Insect-resistant plants further include plants containing at least one transgene comprising a sequence producing upon expression a double-stranded RNA which upon ingestion by a plant insect pest inhibits the growth of this insect pest, as described e.g. in WO 2007/080126, WO 2006/129204, WO 2007/074405, WO 2007/080127 and WO 2007/035650.

Further expression products of interest confer tolerance to abiotic stresses. Plants with such tolerance can be obtained by genetic transformation, or by selection of plants containing a mutation imparting such stress resistance. Particularly useful stress tolerance plants include:
1) plants which contain a transgene capable of reducing the expression and/or the activity of poly(ADP-ribose) polymerase (PARP) gene in the plant cells or plants as described in WO 00/04173, WO/2006/045633, EP 04077984.5, or EP 06009836.5.
2) plants which contain a stress tolerance enhancing transgene capable of reducing the expression and/or the activity of the PARG encoding genes of the plants or plants cells, as described e.g. in WO 2004/090140.
3) plants which contain a stress tolerance enhancing transgene coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase as described e.g. in EP 04077624.7, WO 2006/133827, PCT/EP07/002433, EP 1999263, or WO 2007/107326.

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics and include:
a) Plants, such as cotton plants, containing an altered form of cellulose synthase genes as described in WO 98/00549
b) Plants, such as cotton plants, containing an altered form of rsw2 or rsw3 homologous nucleic acids as described in WO 2004/053219
c) Plants, such as cotton plants, with increased expression of sucrose phosphate synthase as described in WO 01/17333
d) Plants, such as cotton plants, with increased expression of sucrose synthase as described in WO 02/45485
e) Plants, such as cotton plants, wherein the timing of the plasmodesmatal gating at the basis of the fiber cell is altered, e.g. through downregulation of fiber-selective β-1,3-glucanase as described in WO 2005/017157, or as described in EP 08075514.3 or U.S. Patent Appl. No. 61/128,938
f) Plants, such as cotton plants, having fibers with altered reactivity, e.g. through the expression of N-acetylglucosaminetransferase gene including nodC and chitin synthase genes as described in WO 2006/136351

Among the genes which encode proteins or RNAs that confer useful agronomic properties on the transformed plants, mention can further be made of the DNA sequences encoding proteins which confer tolerance to certain insects or those which confer tolerance to certain diseases.

Such genes are in described for example in published PCT Patent Applications WO 91/02071 and W095/06128.

The transformed plant cells and plants described herein such as those obtained by the methods described herein may be further used in breeding procedures well known in the art, such as crossing, selfing, and backcrossing. Breeding programs may involve crossing to generate an F1 (first filial) generation, followed by several generations of selfing (generating F2, F3, etc.). The breeding program may also involve backcrossing (BC) steps, whereby the offspring is backcrossed to one of the parental lines, termed the recurrent parent.

Accordingly, also disclosed herein is a method for producing plants comprising the chimeric gene disclosed herein comprising the step of crossing the cotton plant disclosed herein with another plant or with itself and selecting for offspring comprising said chimeric gene.

The transgenic plant cells and plants obtained by the methods disclosed herein may also be further used in subsequent transformation procedures, e.g. to introduce a further chimeric gene.

The cotton plants or seed comprising the chimeric gene disclosed herein or obtained by the methods disclosed herein may further be treated with cotton herbicides such as Diuron, Fluometuron, MSMA, Oxyfluorfen, Prometryn, Trifluralin, Carfentrazone, Clethodim, Fluazifop-butyl, Glyphosate, Norflurazon, Pendimethalin, Pyrithiobac-sodium, Trifloxysulfuron, Tepraloxydim, Glufosinate, Flumioxazin, Thidiazuron; cotton insecticides such as Acephate, Aldicarb, Chlorpyrifos, Cypermethrin, Deltamethrin, Abamectin, Acetamiprid, Emamectin Benzoate, Imidacloprid, lndoxacarb, Lambda-Cyhalothrin, Spinosad, Thiodicarb, Gamma-Cyhalothrin, Spiromesifen, Pyridalyl, Flonicamid, Flubendiamide, Triflumuron, Rynaxypyr, Beta-Cyfluthrin, Spirotetramat, Clothianidin, Thiamethoxam, Thiacloprid, Dinetofuran, Flubendiamide, Cyazypyr, Spinosad, Spinotoram, gamma Cyhalothrin, 4-[[(6-Chlorpyridin-3-yl)methyl](2,2-difluorethyl)amino]furan-2(5H)-on, Thiodicarb, Avermectin, Flonicamid, Pyridalyl, Spiromesifen, Sulfoxaflor; and cotton fungicides such as Azoxystrobin, Bixafen, Boscalid, Carbendazim, Chlorothalonil, Copper, Cyproconazole, Difenoconazole, Dimoxystrobin, Epoxiconazole, Fenamidone, Fluazinam, Fluopyram, Fluoxastrobin, Fluxapyroxad, Iprodione, Isopyrazam, Isotianil, Mancozeb, Maneb, Metominostrobin, Penthiopyrad, Picoxystrobin, Propineb, Prothioconazole, Pyraclostrobin, Quintozene, Tebuconazole, Tetraconazole, Thiophanate-methyl, Trifloxystrobin. For a treatment with cotton herbicides, said cotton plants or seed preferably further comprise a trait conferring a respective herbicide tolerance.

For the purpose of this invention, the “sequence identity” of two related nucleotide or amino acid sequences, expressed as a percentage, refers to the number of positions in the two optimally aligned sequences which have identical residues (x100) divided by the number of positions compared. A gap, i.e. a position in an alignment where a residue is present in one sequence but not in the other, is regarded as a position with non-identical residues. The alignment of the two sequences is performed by the Needleman and Wunsch algorithm (Needleman and Wunsch 1970). The computer-assisted sequence alignment above, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3.

The sequence listing contained in the file named “BCS11-2004-WO1_ST25”, which is 23 kilobytes (size as measured in Microsoft Windows®), contains 2 sequences SEQ ID NO: 1 and SEQ ID NO: 2, is filed herewith by electronic submission and is incorporated by reference herein.

All patents, patent applications and publications mentioned herein are hereby incorporated by reference, in their entireties, for all purposes.

### FIGURE LEGENDS

FIG. 1: Expression of the GC1 promoter in GC1::GUS transgenic cotton plant.

Leaf tissue from GC1::GUS transgenic TO plant displays a localized expression of the GUS reporter gene in stomata.

Arrows indicate the stomata which are stained blue.

FIG. 2: Relative GUS expression in GC1::GUS transgenic cotton plants as compared to non-transgenic control. Black bars: guard cells. Gray bars: epidermal cells.

The following examples illustrate the invention. It is to be understood that the examples do not limit the spirit and scope of the subject-matter disclosed herein.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R.D.D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### EXAMPLES

Materials

Unless indicated otherwise, chemicals and reagents in the examples were obtained from Sigma Chemical Company, restriction endonucleases were from Fermentas or Roche-Boehringer, and other modifying enzymes or kits regarding biochemicals and molecular biological assays were from Qiagen, Invitrogen and Q-BIOgene. Bacterial strains were from Invitrogen. The cloning steps carried out, such as, for example, restriction cleavages, agarose gel electrophoresis, purification of DNA fragments, linking DNA fragments, transformation of E. coli cells, growing bacteria, multiplying phages and sequence analysis of recombinant DNA, are carried out as described by Sambrook (1989). The sequencing of recombinant DNA molecules is carried out using ABI laser fluorescence DNA sequencer following the method of Sanger.

### Example 1

### Generation of Expression Constructs with a 1719 by Region from the GC1 Promoter (pGC1) Operably Linked to the GUS Reporter Gene

Generation of the Expression Vectors:

The 1719 by promoter of the GC1 gene of Arabidopsis thaliana (5′ to 3′ position 4110 to 2392 of SEQ ID NO: 1), the GUS gene with intron (5′ to 3′ position 2387 to 390 of SEQ ID NO: 1) and a fragment of the 3′ untranslated region (UTR) of the CaMV 35S gene (5′ to 3′ position 313 to 92 of SEQ ID NO: 1) were assembled in a vector which contains the 2mepsps selectable marker cassette (position 6669 to 8006 of SEQ ID NO: 1) to result in expression vector pTCD99 (SEQ ID NO: 2).

### Example 2

### Generation of Transgenic Plants Comprising pGC1::GUS

In a next step the recombinant vector comprising the expression cassettes of example 1, i.e. vector pTCD99, was used to stably transform Gossypium hirsutum coker 312 using an embryogenic callus transformation protocol.

### Example 3

### Stomata-Specific Expression of pGC1::GUS

β-glucuronidase activity of plants transformed with pTCD99 was monitored in planta with the chromogenic substrate X-Gluc (5-bromo-4-Chloro-3-indolyl-β-D-glucuronic acid) during corresponding activity assays (Jefferson R A et al (1987) EMBO J. 20; 6(13):3901-7). For determination of promoter activity plant tissue was dissected, embedded, stained and analyzed as described (e.g., Pien S. et al (2001) PNAS 98(20):11812-7). Thus, the activity of beta-glucuronidase in the transformed plants was witnessed by the presence of the blue color due to the enzymatic metabolism of the substrate X-Gluc.

After growing 30 independent TO plants with sufficient water supply plants were examined for GUS expression. From these plants leaf samples from the first pair of leaves were taken and tested for GUS reporter gene expression (e.g., Pien S. et al (2001) PNAS 98(20):11812-7).

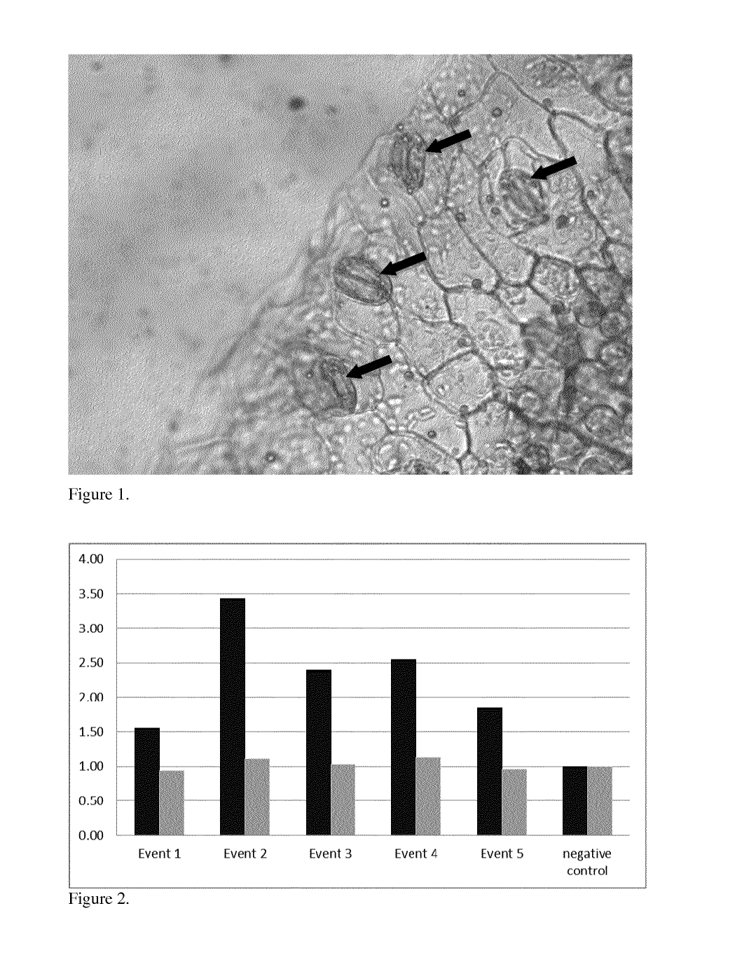

It was observed that GUS was only expressed in the stomata (see FIG. 1).

GUS staining was quantified in either the guard cells or the non-guard cells (epidermal cells) using the tool ImageJ. The staining was corrected for background staining in non-transformed controls by determining the ratio of staining in transgenic event comprising pGC1::GUS over negative control. Table 1 and FIG. 2 show that only the guard cells, and not the epidermal cells show GUS activity over background levels, showing that the GC1 promoter drives guard cell specific expression in cotton leaves.

TABLE 1
GUS staining in guard cells and epidermal cells of different transgenic
events comprising pGC1::GUS (Event s1-5) relative to non-transformed
control. Staining was quantified using the tool ImageJ.

| | guard cells | epidermal cells |
|---|---|---|
| Event 1 | 1.55 | 0.94 |
| Event 2 | 3.43 | 1.11 |
| Event 3 | 2.40 | 1.03 |
| Event 4 | 2.55 | 1.13 |
| Event 5 | 1.85 | 0.96 |
| negative control | 1 | 1 |

### REFERENCES

Ackerson, R. C. (1982). Synthesis and Movement of Abscisic Acid in Water-Stressed Cotton Leaves. Plant Physiol 69, p. 609-613

Carrington & Freed 1990. Cap-independent enhancement of translation by a plant potyvirus 5 nontranslated region. J. Virol. 64, p. 1590-1597

Chaudhary et al. (2008). EVOLUTION & DEVELOPMENT 10(5), p. 567-582

Hubbard, K. E., Nishimura, N, Hitomi, K, Getzoff, E. D. and Julian I. Schroeder (2010). Early abscisic acid signal transduction mechanisms: newly discovered components and newly emerging questions. Genes Dev 24: 1695-1708

Kim T. H., Böhmer M., Hu H., Nishimura N., and Schroeder J. I (2010) Guard Cell Signal Transduction Network: Advances in Understanding Abscisic Acid, CO₂, and Ca²⁺ Signaling. Annu Rev Plant Biol. 61: 561-591

Logan, Edwards and Saunders (Editors; Real-Time PCR: Current Technology and Applications, Caister Academic Press 2009, ISBN: 978-1-904455-39-4

Löpez-Carbonell and Jäuregui (2005). A rapid method for analysis of abscisic acid (ABA) in crude extracts of water stressed Arabidopsis thaliana plants by liquid chromatography-mass spectrometry in tandem mode. Plant Physiol Biochem 43: 407-411

Mansfield, T. A. (1987) Hormones as regulators of water balance. In Plant hormones and their role in growth and development, R. D. Davies, ed (Dordrecht, The Netherlands: Martinus Nijhoff Publishers), p. 411-430

Needleman and Wunsch (1970). A general method applicable to the search for similarities in the amino acid sequence of two proteins. J. Mol. Biol., 48: 443-453

Pandey S., Zhang W., Assmann S.M. (2007). Roles of ion channels and transporters in guard cell signal transduction. FEBS Lett. 581: 2325-36

Pearson and Lipman (1988). Improved tools for biological sequence comparison. Proc. Natl. Acad. Sci 85: 2444-48.

Sambrook, J. F., Russell, D. W. and Irwin, N. (2000). Molecular Cloning: A Laboratory Manual, 3^{rd} edition Volumes 1, 2, and 3. Cold Spring Harbor Laboratory Press

Schroeder J. I., Allen G. J., Hugouvieux V., Kwak J. M., Waner D. (2001). Guard cell signal transduction. Annu Rev Plant Physiol Plant Mol Biol. 52: 627-58

Seo M. and Koshiba, T. (2002). Complex regulation of ABA biosynthesis in plants. Trends Plant Sci. 7 (1): 41-48

Waterman, M. S. (1995). Introduction to Computational Biology: Maps, sequences and genomes. Chapman & Hall. London

Yamaguchi-Shinozaki, K. and Shinozaki, K. (1993). Characterization of the expression of a desiccation-responsive rd29 gene of Arabidopsis thaliana and alanysis of its promoter in transgenic plants. Mol Gen Genet 236: 331-340

Yamaguchi-Shinozaki and Shinozaki (1994). A Novel cis-Acting Element in an Arabisopsis Gene Is Involved in Responsiveness to Drought, Low-Temperature, or High-Salt Stress. The Plant Cell 6: 251-264

Zanoni et al. (2009) Nature 460: 264-269; see also Nature Protocols: mRNA expression analysis by Real-Time PCR; ISSN: 1754-2189

Yang Y., costa A., Leonhardt N., Siegel R. S., Schroeder J. I. (2008). Isolation of a strong Arabidopsis guard cell promoter and its potential as a research tool. Plant Methods 4:6

1. A cotton plant cell comprising a chimeric gene comprising
(a) a first nucleic acid sequence comprising at least 700 consecutive nucleotides of SEQ ID NO: 1 or a nucleic acid sequence having at least 80% sequence identity thereto any of which has stomata preferential promoter activity;
(b) a second nucleic acid sequence encoding an expression product of interest; and optionally
(c) a transcription termination and polyadenylation sequence.

2. The cotton plant cell of claim 1, wherein said expression product of interest is
(i) a protein or peptide which is optionally involved in mediating stomatal aperture or
(ii) an RNA molecule capable of modulating the expression of a gene comprised in said cotton plant, wherein said gene comprised in said cotton plant is optionally involved in mediating stomatal aperture.

3. The cotton plant cell of claim 2, wherein said protein or said gene optionally involved in mediating stomatal aperture is selected from a carbonic anhydrase, an S-type channel, OST1, HT1, a protein involved in mediating ABA responsiveness.

4. The cotton plant cell of claim 2 or 3, wherein said RNA molecule comprises a first and second RNA region wherein
1. said first RNA region comprises a nucleotide sequence of at least 19 consecutive nucleotides having at least about 94% sequence identity to the nucleotide sequence of said gene comprised in said cotton plant;
2. said second RNA region comprises a nucleotide sequence complementary to said 19 consecutive nucleotides of said first RNA region; and
3. said first and second RNA region are capable of base-pairing to form a double stranded RNA molecule between at least said 19 consecutive nucleotides of said first and second region.

5. The cotton plant cell of any one of claims 1 to 4, wherein said first nucleic acid sequence comprises the nucleotide sequence of SEQ ID NO: 1.

6. The cotton plant cell of any one of claims 1 to 5, wherein said first nucleic acid sequence consists of SEQ ID NO: 1.

7. A transgenic cotton plant or seed thereof or a cotton plant part comprising
(a) a chimeric gene comprising
a. a first nucleic acid sequence comprising at least 700 consecutive nucleotides of SEQ ID NO: 1 or a nucleic acid sequence having at least 80% sequence identity thereto any of which has stomata preferential promoter activity;
b. a second nucleic acid sequence encoding an expression product of interest; and
c. a transcription termination and polyadenylation sequence; or
(b) the cotton plant cell according to any one of claims 1 to 6.

8. A method of effecting stomata-preferential or stomata-specific expression of a product of interest in cotton comprising:
(a1) introducing a chimeric gene comprising a first nucleic acid sequence comprising at least 700 consecutive nucleotides of SEQ ID NO: 1 or a nucleic acid sequence having at least 80% sequence identity thereto any of which confers stomata-preferential or stomata-specific expression of said chimeric gene, a second nucleic acid sequence encoding an expression product of interest, and a transcription termination and polyadenylation sequence into a cotton plant and growing the plant; OR
(a2) growing the cotton plant of claim 7 or growing a plant from the seed of claim 7.

9. A method of producing a cotton plant or of increasing the yield of a cotton plant comprising:
introducing or introgressing a chimeric gene comprising a first nucleic acid sequence comprising at least 700 consecutive nucleotides of SEQ ID NO: 1 or a nucleic acid sequence having at least 80% sequence identity thereto any of which has stomata preferential promoter activity, a second nucleic acid sequence encoding an expression product of interest, and a transcription termination and polyadenylation sequence; OR
growing the plant of claim 7 or growing a plant from the seed of claim 7.

10. A method of detecting the expression of a transgene comprising
(a) providing the cotton plant cell of any one of claims 1 to 6 or the plant of claim 7, wherein said expression product of interest is the transgene; and
(b) detecting the expression of the transgene.

11. A method for modulating the water use efficiency of a cotton plant comprising
introducing into a cotton plant a chimeric gene comprising
a. a first nucleic acid sequence comprising at least 700 consecutive nucleotides of SEQ ID NO: 1 or a nucleic acid sequence having at least 80% sequence identity thereto any of which has stomata preferential promoter activity;
b. a second nucleic acid sequence encoding an expression product of interest which is involved in mediating stomatal aperture; and
c. a transcription termination and polyadenylation sequence;
growing said cotton plant.

12. The method of any one of claims 8 to 10, wherein said chimeric gene is the chimeric gene described in any one of claims 1 to 6.

13. Use of
(a) the cotton plant or seed of claim 7;
(b) a chimeric gene comprising
a. a first nucleic acid sequence comprising at least 700 consecutive nucleotides of SEQ ID NO: 1 or a nucleic acid sequence having at least 80% sequence identity thereto any of which has stomata preferential promoter activity;
b. a second nucleic acid sequence encoding an expression product of interest; and optionally
c. a transcription termination and polyadenylation sequence; or
(c) a nucleic acid sequence comprising at least 700 consecutive nucleotides of SEQ ID NO: 1 or a nucleic acid sequence having at least 80% sequence identity thereto any of which has stomata preferential promoter activity;
for stomata-preferential expression of a transgene in cotton, for modulating the water use efficiency of a cotton plant or for increasing cotton yield.

14. The use for stomata-preferential expression of claim 13, wherein said chimeric gene is the chimeric gene described in any one of claims 1 to 6.


SEQUENCE LISTING (2 sequences)

SEQ ID NO: 1 - DNA, 1719 bp, Artificial Sequence
  synthetic guard cell promoter
gtatttcttgagtagtgattttgaagtagtgtgtgaaaatagtacttgtggatgattctttatatagatacatttcagctactttttattttctgaacaattactcatgttggatagagatcacatgcatagactttcatcgctggagccaaacataaatcacaggtttttattggataaatcacagattatagattgcatttgctgtaaagtgttggagtttgatatgcaaacagttttgattggttcccttttttcggaggatgcatggaagaagaagaagaaaattcaaacctagtttcactaatatcttaagctaactcaatttgttacgtggaaatgagagtaatttttacattagcttgatactttggtagtttatgtcttaatgatagtttacagctgtctcttagttcagacattgtgacactgagagagatagatgaaggaaacgcagacgcaatattgttcgaaaacacagatgtagtgtttggcgtttaattgtctattttatacaaacgtcattagaaattttctaacaaacatgaaacgtcgaactgaaaacaatatcgcatttgtattggtgttttcataggtttctttaattttaatatatgtatacgagaacacttttagttttcatattggatagaaaaataaaagagaaaaactctacaagacaagtcaaaatgtttgtgttactaaatataaagacaaagcacagcgtgcaaattgctaaagtctacattaattgtgagggagaaaggaaaatgacccgagagggagacatcaatttaatgacaattttgattaaaaggcttataaccaatgaaaatattaatgtcctcaaaatgggtcagatccaataagcggcacccttcattaggcccatctatatttgagaaaatttatcatctacaattctacatcgtcaattcctaaagattgattgtataggttttttttcttgtttcataagaattattctttcttttttttatgtcttatgcatatttgttaatgttagtgagtcacaaaatttagatatttgaattcactaaccattttaccaaaaataaaaatttactccactatagattgttgtccgtctaataagaaaagcggtgttgaaaacttataaattcatcctctctgttgcaaccattttgtttcttatgttgacaagtttgtcctacacgtccaaagttttatataaaagagatcaaagagacaacaagttctcaagtccaaaactaaacactattttgaatggatacttcgccaaggacccgaattgtacctactagacgatccaactcggtcaaattctcctcgacggttaacatttcaacccggcgatcactctcttcctcctcctccaattccctcctattctccggcaactctccggcaactcctctccatccacttggcatcccattttcatgggaaaagcttcccggtaaacctaaagagttctcgtatcaccggaaaaacgaatcattaagcaacctcttgcctctccctcctcactggagtaaccatagccctaacaccagatgtaagaagaacacgtcccctgcggccatgagagatcctttcatggcggcgttggtagagtgttccaaaggcgctaccacagacggagaacataccggcggcgaatcaaggaaggtcttgaggaagagcaggagcatcgggttactgaatctttactcg

SEQ ID NO: 2 - DNA, 15137 bp, Artificial Sequence
  expression vector pTDC99
aattacaacggtatatatcctgccagtactgggccccctcgagggcgatcgctacgtacctgcaggcaattggccgctgtaccatgcatgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaatagagagagatagatttgtagagagagactggtgatttcagcgtgtccaagcttgctagtccccgagctcggtagcaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggaccatggtatttcttgagtagtgattttgaagtagtgtgtgaaaatagtacttgtggatgattctttatatagatacatttcagctactttttattttctgaacaattactcatgttggatagagatcacatgcatagactttcatcgctggagccaaacataaatcacaggtttttattggataaatcacagattatagattgcatttgctgtaaagtgttggagtttgatatgcaaacagttttgattggttcccttttttcggaggatgcatggaagaagaagaagaaaattcaaacctagtttcactaatatcttaagctaactcaatttgttacgtggaaatgagagtaatttttacattagcttgatactttggtagtttatgtcttaatgatagtttacagctgtctcttagttcagacattgtgacactgagagagatagatgaaggaaacgcagacgcaatattgttcgaaaacacagatgtagtgtttggcgtttaattgtctattttatacaaacgtcattagaaattttctaacaaacatgaaacgtcgaactgaaaacaatatcgcatttgtattggtgttttcataggtttctttaattttaatatatgtatacgagaacacttttagttttcatattggatagaaaaataaaagagaaaaactctacaagacaagtcaaaatgtttgtgttactaaatataaagacaaagcacagcgtgcaaattgctaaagtctacattaattgtgagggagaaaggaaaatgacccgagagggagacatcaatttaatgacaattttgattaaaaggcttataaccaatgaaaatattaatgtcctcaaaatgggtcagatccaataagcggcacccttcattaggcccatctatatttgagaaaatttatcatctacaattctacatcgtcaattcctaaagattgattgtataggttttttttcttgtttcataagaattattctttcttttttttatgtcttatgcatatttgttaatgttagtgagtcacaaaatttagatatttgaattcactaaccattttaccaaaaataaaaatttactccactatagattgttgtccgtctaataagaaaagcggtgttgaaaacttataaattcatcctctctgttgcaaccattttgtttcttatgttgacaagtttgtcctacacgtccaaagttttatataaaagagatcaaagagacaacaagttctcaagtccaaaactaaacactattttgaatggatacttcgccaaggacccgaattgtacctactagacgatccaactcggtcaaattctcctcgacggttaacatttcaacccggcgatcactctcttcctcctcctccaattccctcctattctccggcaactctccggcaactcctctccatccacttggcatcccattttcatgggaaaagcttcccggtaaacctaaagagttctcgtatcaccggaaaaacgaatcattaagcaacctcttgcctctccctcctcactggagtaaccatagccctaacaccagatgtaagaagaacacgtcccctgcggccatgagagatcctttcatggcggcgttggtagagtgttccaaaggcgctaccacagacggagaacataccggcggcgaatcaaggaaggtcttgaggaagagcaggagcatcgggttactgaatctttactcggcgcgcccccgatccgcgtttgtgttttctgggtttctcacttaagcgtctgcgttttacttttgtattgggtttggcgtttagtagtttgcggtagcgttcttgttatgtgtaattacgctttttcttcttgcttcagcagtttcggttgaaatataaatcgaatcaagtttcactttatcagcgttgttttaaattttggcattaaattggtgaaaattgcttcaattttgtatctaaatagaagagacaacatgaaattcgacttttgacctcaaatcttcgaacatttatttcctgatttcacgatggatgaggataacgaaagggcggttcctatgtccgggaaagttcccgtagaagacaatgagcaaagctactgaaacgcggacacgacgtcgcattggtacggatatgagttaaaccgactcaattcctttattaagacataaaccgattttggttaaagtgtaacagtgagctgatataaaaccgaaacaaaccggtacaagtttgattgagcaacttgatgacaaacttcagaattttggttattgaatgaaaatcatagtctaatcgtaaaaaatgtacagaagaaaagctagagcagaacaaagattctatattctggttccaatttatcatcgctttaacgtccctcagatttgatcggggaattcgatatcattaccctgttatccctaaagcttattaatataacttcgtatagcatacattatacgaagttatgtttgtcgaggagaaatatgagtcgaggcatggatacactaagttcccctgaagtgagcatgatctttgatgctgagatgattcccagagcaagatagtttgtgctgcaagtgacacaattgtaatgaaaccaccactcaacgaatttacttgtggctttgacatgtcgtgtgctctgtttgtatttgtgagtgccggttggtaattatttttgttaatgtgattttaaaacctcttatgtaaatagttactttatctattgaagtgtgttcttgtggtctatagtttctcaaagggaaattaaaatgttgacatcccatttacaattgataacttggtatacacaaactttgtaaatttggtgatatttatggtcgaaagaaggcaatacccattgtatgttccaatatcaatatcaatacgataacttgataatactaacatatgattgtcattgtttttccagtatcaatatacattaagctactacaaaattagtataaatcactatattataaatctttttcggttgtaacttgtaattcgtgggtttttaaaataaaagcatgtgaaaattttcaaataatgtgatggcgcaattttattttccgagttccaaaatattgccgcttcattaccctaatttgtggcgccacatgtaaaacaaaagacgattcttagtggctatcactgccatcacgcggatcactaatatgaaccgtcgattaaaacagatcgacggtttatacatcattttattgtacacacggatcgatatctcagccgttagatttaatatgcgatctgattgctcaaaaaatagactctccgtctttgcctataaaaacaatttcacatctttctcacccaaatctactcttaaccgttcttcttcttctacagacatcaatttctctcgactctagaggatccaagcttatcgatttcgaacccctcaggcgaagaacaggtatgatttgtttgtaattagatcaggggtttaggtctttccattactttttaatgttttttctgttactgtctccgcgatctgattttacgacaatagagtttcgggttttgtcccattccagtttgaaaataaaggtccgtcttttaagtttgctggatcgataaacctgtgaagattgagtctagtcgatttattggatgatccattcttcatcgtttttttcttgcttcgaagttctgtataaccagatttgtctgtgtgcgattgtcattacctagccgtgtatcgagaactagggttttcgagtcaattttgccccttttggttatatctggttcgataacgattcatctggattagggttttaagtggtgacgtttagtattccaatttcttcaaaatttagttatggataatgaaaatccccaattgactgttcaatttcttgttaaatgcgcagatcacaatggcttcgatctcctcctcagtcgcgaccgttagccggaccgcccctgctcaggccaacatggtggctccgttcaccggccttaagtccaacgccgccttccccaccaccaagaaggctaacgacttctccacccttcccagcaacggtggaagagttcaatgtatgcaggtgtggccggcctacggcaacaagaagttcgagacgctgtcgtacctgccgccgctgtctatggcgcccaccgtgatgatggcctcgtcggccaccgccgtcgctccgttccaggggctcaagtccaccgccagcctccccgtcgcccgccgctcctccagaagcctcggcaacgtcagcaacggcggaaggatccggtgcatggccggcgccgaggagatcgtgctgcagcccatcaaggagatctccggcaccgtcaagctgccggggtccaagtcgctttccaaccggatcctcctactcgccgccctgtccgaggggacaacagtggttgataacctgctgaacagtgaggatgtccactacatgctcggggccttgaggactcttggtctctctgtcgaagcggacaaagctgccaaaagagctgtagttgttggctgtggtggaaagttcccagttgaggatgctaaagaggaagtgcagctcttcttggggaatgctggaatcgcaatgcggtccttgacagcagctgttactgctgctggtggaaatgcaacttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggtcaagctgtctggctccatcagcagtcagtacttgagtgccttgctgatggctgctcctttggctcttggggatgtggagattgaaatcattgataaattaatctccattccgtacgtcgaaatgacattgagattgatggagcgttttggtgtgaaagcagagcattctgatagctgggacagattctacattaagggaggtcaaaaatacaagtcccctaaaaatgcctatgttgaaggtgatgcctcaagcgcaagctatttcttggctggtgctgcaattactggagggactgtgactgtggaaggttgtggcaccaccagtttgcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacgtggcttcctggagagtaaaggagaccgagaggatggttgcgatccggacggagctaaccaagctgggagcatctgttgaggaagggccggactactgcatcatcacgccgccggagaagctgaacgtgacggcgatcgacacgtacgacgaccacaggatggcgatggctttctcccttgccgcctgtgccgaggtccccgtcaccatccgggaccctgggtgcacccggaagaccttccccgactacttcgatgtgctgagcactttcgtcaagaattaagctctagaactagtggatcccccgatccgcgtttgtgttttctgggtttctcacttaagcgtctgcgttttacttttgtattgggtttggcgtttagtagtttgcggtagcgttcttgttatgtgtaattacgctttttcttcttgcttcagcagtttcggttgaaatataaatcgaatcaagtttcactttatcagcgttgttttaaattttggcattaaattggtgaaaattgcttcaattttgtatctaaatagaagagacaacatgaaattcgacttttgacctcaaatcttcgaacatttatttcctgatttcacgatggatgaggataacgaaagggcggttcctatgtccgggaaagttcccgtagaagacaatgagcaaagctactgaaacgcggacacgacgtcgcattggtacggatatgagttaaaccgactcaattcctttattaagacataaaccgattttggttaaagtgtaacagtgagctgatataaaaccgaaacaaaccggtacaagtttgattgagcaacttgatgacaaacttcagaattttggttattgaatgaaaatcatagtctaatcgtaaaaaatgtacagaagaaaagctagagcagaacaaagattctatattctggttccaatttatcatcgctttaacgtccctcagatttgatcgggaaacataacttcgtatagcatacattatacgaagttatcaaaacgtcgtgagacagtttggttaactataacggtcctaaggtagcgatcgaggcattacggcattacggcactcgcgagggtccgaatctatgtcgggtgcggagaaagaggtaatgaaatggcaatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaagtgctgtgcacggatctgccctggcttcaggagatcggaagacctcggccgtccgggcgcttgccggtggtgctgaccccggatgaagtctctagagctctagagggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagggatcgataccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgccagattcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcgacacgcgaca